(19) United States
(12) Patent Application Publication
(10) Pub. No.: US 2011/0004138 A1
(43) Pub. Date: Jan. 6, 2011

(54) MEDICAL AID

(76) Inventors: Paavo Lasse Freijzer, Amsterdam (NL); Patrick Johan Gerard Maria Peffer, Nijmegen (NL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/296,824

(22) PCT Filed: Apr. 13, 2007
(86) PCT No.: PCT/NL07/50155

§ 371 Date: Jul. 23, 2010

(30) Foreign Application Priority Data
Apr. 13, 2006 (NL) 1031592

(51) Int. Cl. A61F 5/05

(52) U.S. Cl. 602/22

(57) ABSTRACT

An aid for relative positioning of a palm of hand, associated fingers and forearm, which aid comprises a palm support face for laying against it an inner side of the palm, and a finger support face for laying against it the fingers at least from a point near the metacarpophalangeal joints to a point near the proximal interphalangeal joints.

The present invention relates to a medical aid.

In case of an injury of the hand, such as a broken metacarpus, which requires the palm of the hand, fingers and forearm to be fixated, for instance with plaster of Paris, then, at fixation, the consequences of the position in which fixation is effected are taken into account so as to achieve an optimum medical result and not to prolong a duration of rehabilitation. In particular, palm, fingers and forearm are fixated relative to each other, such that the palm is bent backwards with respect to the forearm, and the fingers in extended condition are positioned transversely to the palm. Thus, muscles and tendons running from the forearm via the palm into the fingers are fixated in a maximally stretched position. During prolonged immobilization of palm, fingers and forearm, a shortening of muscles and/or tendons occurs. By performing the fixation in the above-described manner, this effect of shortening during the period when the patient wears the plaster is prevented to a far-reaching extent, and only little (if any) lengthening needs to be achieved anymore during rehabilitation. This is beneficial to the medical result, with a relatively short period of rehabilitation.

A problem in applying plaster for fixating the palm, fingers and forearm in the above-described fixation position results from the fact that the plaster needs some time to set. During this time, a deviation with respect to this optimum fixation position can occur. Further, properly modeling the plaster requires some experience on the part of the person applying the plaster cast. When the palm and fingers are not fixated in the optimum relative position, this may lead to a longer rehabilitation period, or even entail the necessity of a (restorative) operation. This can be prevented by re-applying plaster in an attempt yet to achieve the optimum fixation position, but history then repeats itself and problems can occur again. This additional work evidently entails additional costs as well.

The object of the present invention is to provide a solution to the above-mentioned problem.

To that end, the present invention provides an aid for relative positioning of a palm of a hand, fingers, and forearm, which aid comprises a palm support face for laying against it the inner side of the palm, and a finger support face for laying against it the fingers at least from a point near the metacarpophalangeal joints to a point near the proximal interphalangeal joints, for instance to a point beyond the proximal interphalangeal joints.

The aid can be used together with a plaster sleeve which in use is arranged at least partly around the palm, fingers and the distal forearm. Then, during the setting of the plaster sleeve, the aid can lie against an outer side of the plaster sleeve remote from the hand. It is also possible that during the setting of the plaster sleeve the aid is placed between the plaster sleeve, the palm, the finger and the distal forearm.

Such an aid makes it possible, in case of a forearm with an injury, in particular an injury of the hand, to bring the fingers, palm and wrist in a desired optimum position and to keep it so during the time the plaster needs to set. In the present application, the term “plaster” should be taken in the widest sense, that is, the term encompasses both classic calcium sulfate-based plaster of Paris, and synthetic plaster, for instance based on a resin. Preferably, the proximal interphalangeal joints of the hand are provided with plaster. Preferably, however, it is ensured that the fingers are fully extended and the fingers are supported also near the ends. In practice, the plaster is not necessarily applied circularly, but may also take the form of a strip which is held in place with a tape. In an embodiment, the palm support face is arranged for supporting substantially the whole palm, and the finger support face is arranged for supporting the four fingers at least to a point near the proximal interphalangeal joints. This embodiment is suitable, for instance, for immobilizing the metacarpals. This embodiment is also designated hereinbelow as four-finger embodiment.

In an alternative embodiment, the palm support face is arranged for supporting the inner side of the palm under substantially exclusively the fourth and fifth metacarpals, and the finger support face is arranged for supporting substantially exclusively the little finger and ring finger at least to a point near the proximal interphalangeal joints. This embodiment is suitable, for instance, for immobilizing the metacarpus of the fifth radius (associated with the little finger), for instance in case of a fracture of that metacarpus. This embodiment is also referred to hereinbelow as two-finger embodiment.

Preferably, the transition of the palm support face and the finger support face comprises a first knuckle support surface and a second knuckle support surface situated remote from the first knuckle support surface, the first knuckle support surface of the transition being situated higher than the second knuckle support surface.

In the four-finger embodiment, the first knuckle support surface can then serve for supporting the area of the hand under the knuckle of the index finger and/or ring finger, and the second knuckle support surface can serve for supporting the area of the hand under the knuckle of the little finger. Thus, the hand is supported in an ergonomically sound manner. The term knuckle is here understood to mean the metacarpophalangeal joint.

According to a particular embodiment of this, between the first knuckle support surface and the second knuckle support surface of the transition, a third knuckle support surface is provided, which is situated higher than the first knuckle support surface. The third knuckle support surface can then serve for supporting the area of the hand under the knuckle of the middle finger. Also, the knuckle support surfaces of the transition from the palm support face to the finger support face are preferably rounded so as to avoid a sharp angle adjacent the transition joints (knuckles) between palm and fingers. Thus, the aid and/or the plaster can afford an optimum, uniform support and the pressure distribution can be as uniform as possible.

In the two-finger embodiment, the first knuckle support surface can serve for supporting the area of the hand under the knuckle of the little finger, and the second knuckle support surface can serve for supporting the area of the hand under the knuckle of the ring finger. Thus, the hand is supported in an ergonomically sound manner.

Preferably, the palm support face and the finger support face include a mutual angle of 75-110°, preferably 85-95°. Thus, the fingers and the palm can be brought in a desired optimum position, for instance fixated, for instance during the time the plaster needs to set. Thus, an optimally stretched position of the tendons in fingers, hand and forearm is achieved.

In an embodiment, the palm support face is made convex over at least a part of the surface thereof. Thus, the palm is loaded more uniformly in that the pressure is distributed uniformly over the palm, which enhances comfort for the patient.

In an embodiment, the finger support face is made convex over at least a part of the surface thereof. Thus, the fingers are loaded more uniformly in that the pressure is distributed uniformly over the fingers, which enhances comfort for the patient.

In principle, with an aid according to the invention, the wrist, or more generally, the forearm, can rest on the same surface as that on which the aid has been placed or secured. However, preferably, the aid is characterized in that the aid, remote from the transition of the palm support face and the finger support face, on the side of the palm support face, possesses a forearm support face for positioning the forearm, the palm support face and the forearm support face including a mutual angle of 65-100°.

Thus, the wrist, or more generally the forearm, can be given support, for instance during the setting of the plaster, such that a desired angle between hand and wrist is maintained during the setting of the plaster. The forearm support face makes it possible, when such is desired, to provide in a simple manner an angle with the surface on which the aid has been placed. This angle will generally be less than 10°.

Preferably, the palm support face and the forearm support face include a mutual angle of 65-85°. Such an angle is optimal for ensuring that an optimum stretched position of the tendons in fingers, hand and wrist is achieved.

Preferably, the forearm support face is made of lowered design in the width direction near the middle thereof. Thus, the forearm support face is adapted to the shape of the forearm.

Preferably, the aid is of one-piece design. This provides the advantage that the aid can be made of simple design, for instance free of settings, so that the aid can be simply applied, for instance by staff with only a limited instruction, while a risk that the aid will be wrongly applied to the palm, fingers and possibly forearm is limited.

Preferably, the palm support face, the finger support face and possibly the forearm support face of the aid are designed as a substantially plate-shaped body, preferably the aid is designed as a substantially plate-shaped body. This provides the advantage that the aid can be manufactured in a very simple and inexpensive manner. The aid may then be designed, for instance, as a disposable article. Preferably, the substantially plate-shaped body is of a thickness of 1-10 mm, preferably 2-7 mm, more preferably 3-5 mm. Preferably, the substantially plate-shaped body is manufactured from metal and/or plastic. Preferably, the aid is manufactured by injection molding, thermoforming, deep-drawing and/or deep-pressing. The invention also relates to a method for applying plaster to a hand, comprising applying a plaster sleeve, at least partly, around the hand, laying an aid according to the invention against the hand, and allowing the plaster sleeve to set while the plaster sleeve lies against the aid. Here, applying the plaster sleeve, at least partly, around the hand is understood to mean applying the plaster sleeve, at least partly, around the fingers, the palm and the distal forearm. Such a method makes it possible, when applying plaster to the hand, to bring the fingers, palm, and the wrist in a desired optimum position and to keep this so during the time the plaster needs to set.

In an embodiment, during the setting of the plaster, the aid lies against an outer side of the plaster sleeve remote from the hand. This provides the advantage that the plaster can set while the aid keeps the plaster and the hand in the proper position. Then, if desired, after the plaster has set, the aid can be removed and optionally be reused

In an alternative embodiment, during the setting of the plaster, the aid is placed between the hand and the plaster sleeve. Here, the plaster sleeve can be arranged, at least partly, around the aid and the hand, such that the aid, by means of the plaster sleeve, is permanently connected with the hand, that is, the aid can be removed from the hand exclusively by damaging the plaster sleeve and/or the aid. The aid can thus keep the hand in the proper position during the whole time that the plaster is connected with the hand. If so desired, the aid can then be designed as a disposable article which, after removal of the plaster and the aid from the hand, can be thrown away.

The present invention further relates to a method for treating an individual with an injury of a forearm, understood to encompass a hand of the forearm, which requires the hand to be fixated in a position such that the tendons and/or muscles of fingers, palm and/or forearm are in a stretched condition utilizing plaster, wherein the fingers, palm and forearm are provided with non-set plaster, and at least the fingers and the palm of the hand are laid against a finger support face and a palm support face, respectively, of an aid according to the invention, until the plaster has set sufficiently. The plaster is in the form of a strip (in which case it will be held in place by a tape), but can also be applied circularly at least over a part of the length thereof. Preferably, an aid according to any one of the subclaims is used. If a forearm support face is present, the forearm is preferably placed in contact with the forearm support face.

The present invention will now be elucidated with reference to the drawing, wherein

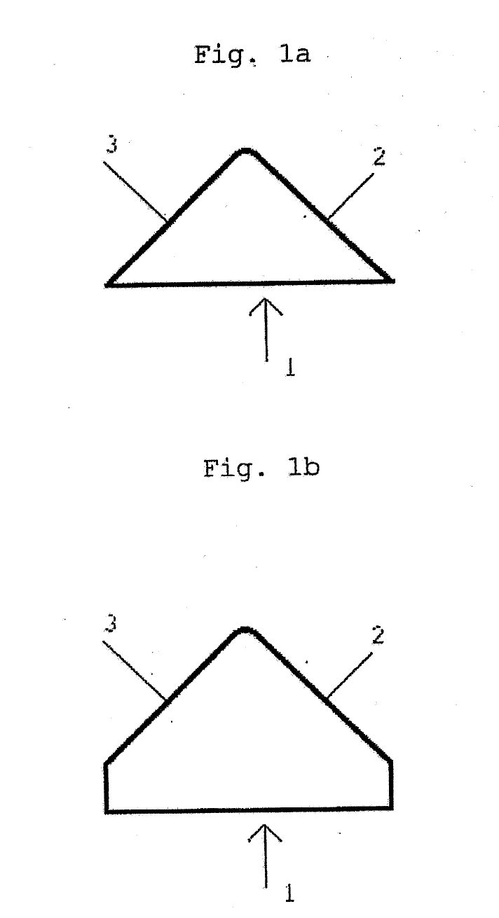

FIG. 1a shows a side elevation of an aid according to the invention, and

FIG. 1b shows a variant thereof;

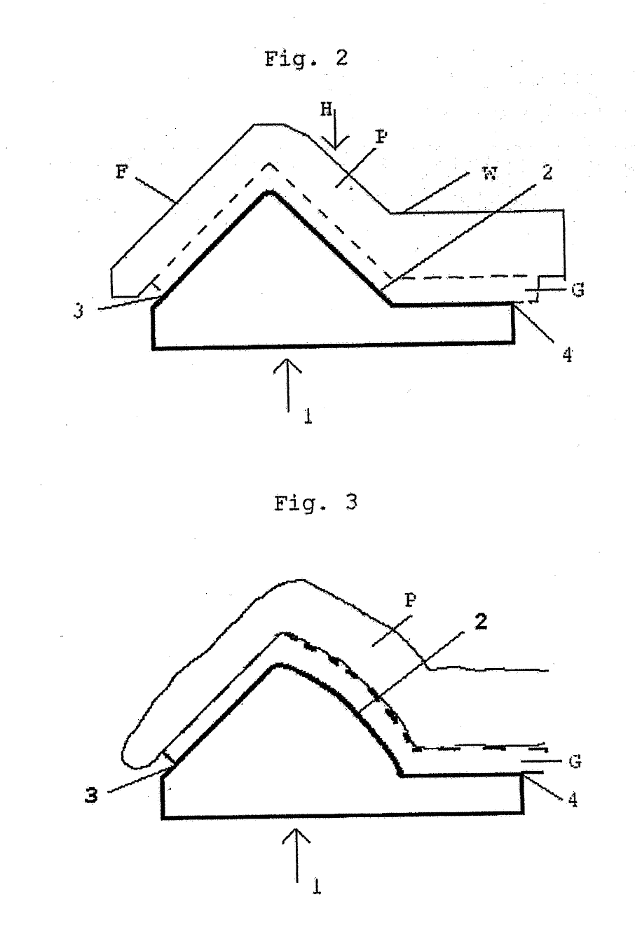

FIG. 2 shows a further variant of the aid according to the invention, during use of the aid;

FIG. 3 represents an alternative embodiment of the aid depicted in FIG. 2;

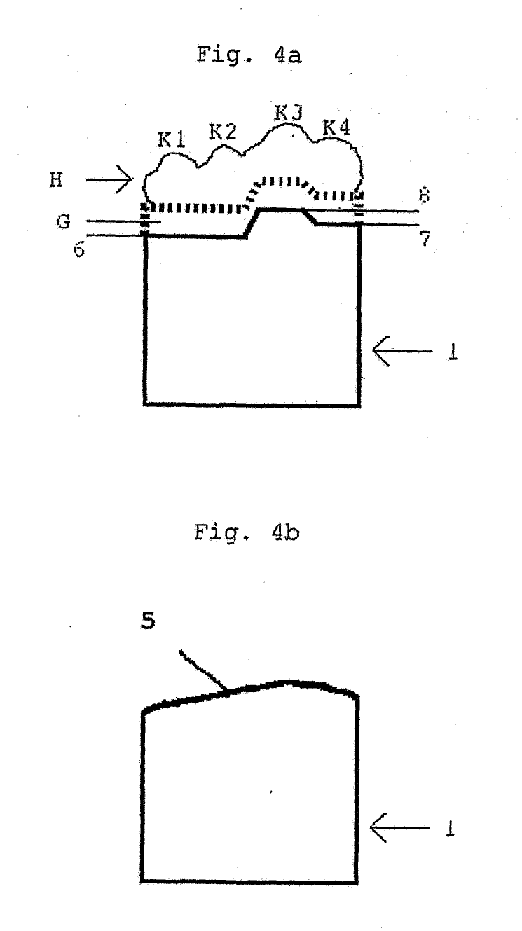

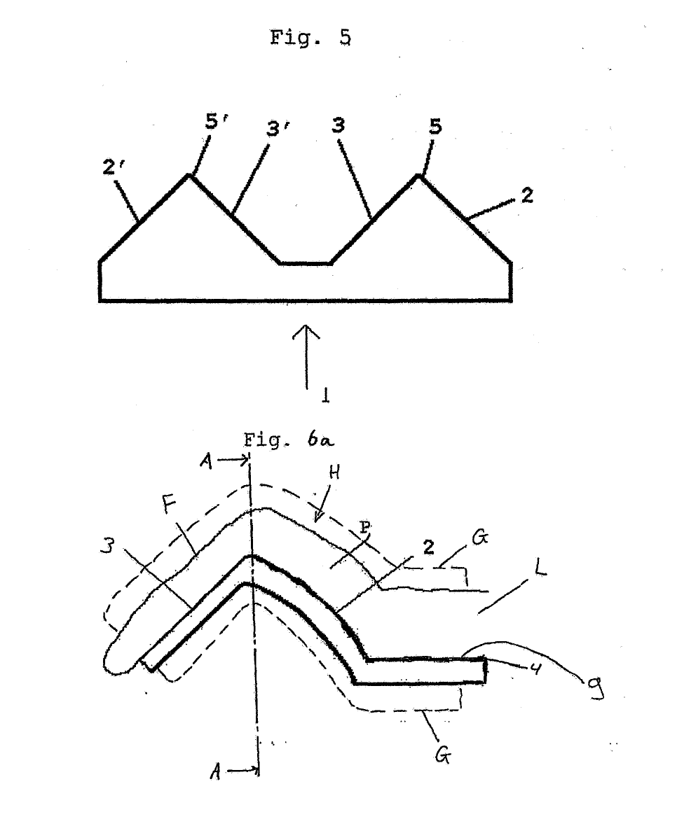

FIG. 4a schematically shows a front view of the aid depicted in FIG. 1a; and FIG. 4b shows a more ergonomic variant thereof;

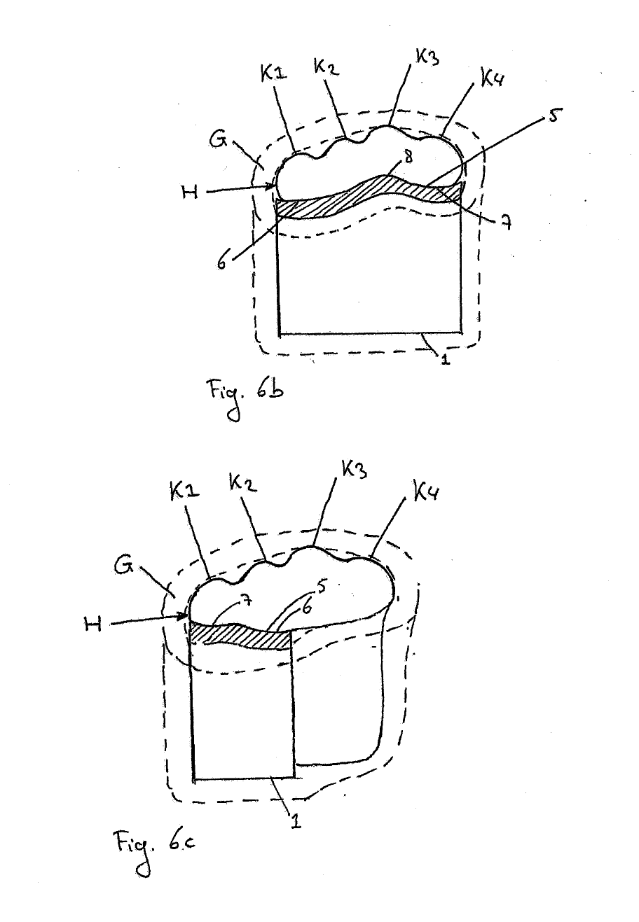

FIG. 5 shows a variant of the embodiment represented in FIG. 1b;

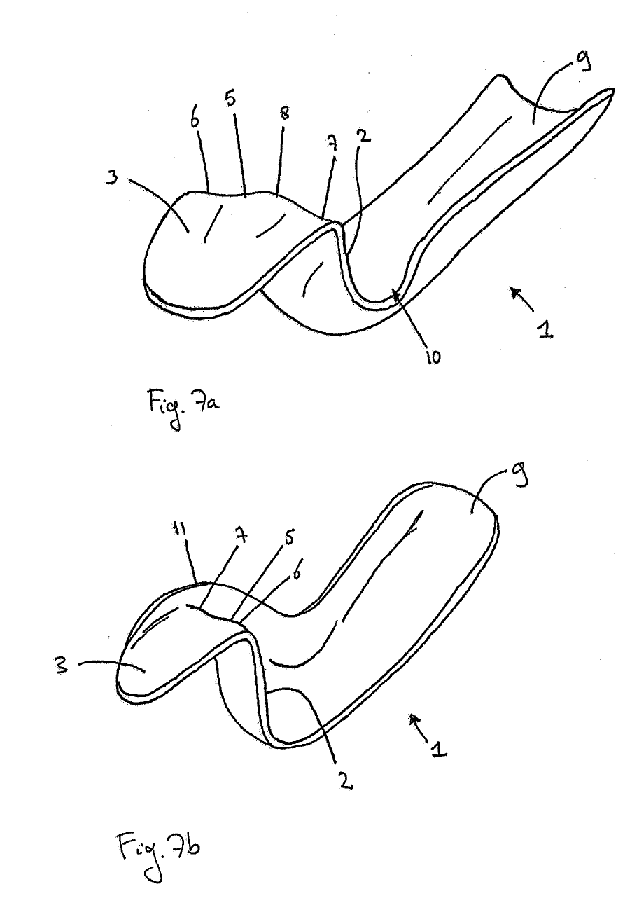

FIG. 6a shows a further variant of the aid according to the invention, during use of the aid;.

FIG. 6b shows a view in cross section along the line A-A of the aid shown in FIG. 6a;

FIG. 6c shows a variant of the aid shown in FIGS. 6a and 6b;

FIG. 7a shows an advanced embodiment of the aid according to the invention; and

FIG. 7b shows an alternative advanced embodiment of the aid according to the invention.

FIG. 1a shows a side elevation of an aid 1 for relative positioning of a palm of a hand, fingers, and wrist, as is necessary, in case of an injury such as a broken metacarpus, in applying plaster, and for the setting thereof. FIG. 1b and FIG. 2 show two variants of the aid. The hand, designated by the reference sign H (FIG. 2), possesses a palm P and fingers F. The wrist is designated by the reference sign W. The aid 1 possesses a first support face 2, hereinafter designated as palm support face, which serves for applying the palm P of the hand H, and a second support face 3, hereinafter designated as finger support face, which serves for applying the fingers F of the hand H. Before the hand H is laid against the aid 1, in this example, the hand H is provided with plaster G, which is represented with a broken line. Before the plaster G has set, the hand H provided with plaster is laid against the aid 1 for proper relative positioning of the fingers F, palm P and wrist W. As represented in FIGS. 2 and 3, the aid 1 can possess a wrist support 4, provided with a third support face 9, hereinafter designated as forearm support face, for positioning the forearm L against it. This is considered favorable for better positioning of the wrist W, but is not strictly necessary, since the wrist W can also rest on the supporting surface on which the aid 1 has been placed. Instead, proper positioning of the wrist W relative to the palm F can also be achieved by lifting the elbow. That supporting surface can for instance be formed by a plaster table. As soon as a sufficient strength of the plaster G has been achieved, usually after a few minutes, the hand H can be taken off the aid and the plaster G can set further without contact with the aid 1.

The height from the underside (base) of the aid 1 to the highest point (i.e. the transition 5) depends on the size of the patient's hand. A relatively high aid is also suitable for patients with small hands, certainly when use is made of a wrist support 4 (which may or may not be detachable). The height is for instance between 5 and 10 cm.

The angle between the palm support face 2 and the finger support face 3 is 90° in the embodiment represented, which angle is considered ideal.

FIG. 3 essentially corresponds to FIG. 1, except that here the palm support face 2 is of convex (spherical) design, such that a uniform pressure is exerted on the plaster G which is present at the palm P. This reduces discomfort for the patient. The palm support face can then comprise a convexity in a longitudinal direction of the palm and/or in the width direction of the palm. It is not requisite that the convexity in the longitudinal direction is equal to the convexity in the width direction.

It is also possible that the finger support face 3 is made of convex (spherical) design, such that a uniform pressure is exerted on the plaster G which is present at the fingers F. This reduces discomfort for the patient. The finger support face can then comprise a convexity in a longitudinal direction of the fingers and/or in the width direction of the fingers. It is not requisite that the convexity in the longitudinal direction is equal to the convexity in the width direction.

FIG. 4a shows a schematic front view of the aid 1 for a right hand H (which hand is shown in cross section). It can be seen that at the transition 5 between the palm support face 2 and the finger support face 3, three knuckle support surfaces of the transition are indicated, where the knuckle K1 of the little finger of the right hand H will be laid against a second knuckle support surface 6 of the transition 5, and the knuckle K4 of the index finger against a first knuckle support surface 7. The first knuckle support surface 7 is situated higher than the second knuckle support surface 6. A third knuckle support surface 8, contiguous to the first knuckle support surface 7, is situated higher than the first knuckle support surface 7. The knuckle K3 of the middle finger of the hand H will be laid against this third knuckle support surface 8 of the transition 5. When in this application, reference is made to applying or laying a part of the body against the aid, this is understood to encompass applying or laying that part of the body against the aid while the respective part of the body is separated from the aid 1 by plaster and/or possibly a cushion layer such as cotton wadding (not shown). FIG. 4 shows an ergonomic variant of the aid 1 for a right hand, where the transition 5 is of smoothly curved design, so as to follow the knuckle arc of the palm as smoothly as possible.

It will be clear that in FIG. 4b likewise the second knuckle support surface 6 is situated at the location where the knuckle K1 of the little finger of the right hand H will be laid against the transition 5, the first knuckle support surface 7 is situated at the location where the knuckle K4 of the index finger will be laid against the transition 5, and the third knuckle support surface 8 is situated at the location where the knuckle K3 of the middle finger will be laid against the transition 5. The first knuckle support surface 7 is then situated higher than the second knuckle support surface 6, and the third knuckle support surface 8 is situated higher than the first knuckle support surface 7.

FIG. 5 shows a possible embodiment suitable for both a left hand and a right hand. The reference numerals for the aid for positioning the left hand are indicated with an accent. Instead, the aids for the left hand and the right hand can also be placed with the transitions 5, 5′ next to or near each other, with the transitions 5, 5′ being in line with each other.

FIG. 6a shows a further variant of the aid 1 according to the invention, during use of the aid. In FIG. 6a, the palm P of the hand H lies against the palm support face 2, and the fingers F lie against a finger support face. In this example, furthermore, the forearm L lies against the forearm support face 9. In this example, first the hand is laid against the aid 1, and then the hand is provided with plaster G, which is represented with a broken line. In this example, the plaster encloses the hand, in this example the palm P, at least a part of the fingers F and at least a part of the forearm L, like a plaster sleeve. In this example, the plaster is provided, at least partly, around the aid 1 and the hand, such that the aid, by means of the plaster, is permanently connected with the hand. The plaster will thus set, while the hand lies against the aid and is so held in the proper position. In the example of FIG. 6, the aid 1 is of substantially plate-shaped design. This makes it possible for the plaster to be applied to the hand and/or forearm in a conventional manner.

It will be clear that in the example of FIG. 6a, the palm support face 2 and/or the finger support face 3 can have a convexity as described with reference to FIG. 3.

FIG. 6b shows a view in cross section along the line A-A of the aid shown in FIG. 6a. In the example of FIG. 6a, it can be seen that the plaster G forms a plaster sleeve around the hand adjacent the knuckles. It will be clear that in FIG. 6b likewise the second knuckle support surface 6 is situated at the location where the knuckle K1 of the little finger of the right hand H will be laid against the transition 5, the first knuckle support surface 7 is situated at the location where the knuckle K4 of the index finger will be laid against the transition 5, and the third knuckle support surface 8 is situated at the location where the knuckle K3 of the middle finger will be laid against the transition 5. Also in FIG. 6b the first knuckle support surface 7 is situated higher than the second knuckle support surface 6, and the third knuckle support surface 8 is situated higher than the first knuckle support surface 7.

FIG. 6c shows a variant of the aid 1 shown in FIGS. 6a and 6b, with the palm support face 2 being arranged for supporting the inner side of the palm under substantially exclusively the fourth and fifth metacarpals, associated with the ring finger and the little finger, respectively, and the finger support face 3 being arranged for supporting substantially exclusively the little finger and ring finger at least to a point near the proximal interphalangeal joints. This embodiment is for instance suitable for immobilizing the metacarpus associated with the little finger, for instance in case of a fracture of that metacarpus.

FIG. 7a shows an advanced embodiment of the aid 1 according to the invention. In this embodiment, the palm support face 2 is arranged for supporting substantially the whole palm, and the finger support face 3 is arranged for supporting the four fingers at least to a point near the proximal interphalangeal joints, in this example to a point beyond the proximal interphalangeal joints. In this example, the aid 1 is designed in one piece, i.e. of monolithic design. In this example, the aid 1 is designed as a substantially plate-shaped body. The aid can for instance be obtained by deforming, for instance thermoforming or vacuum forming, a planar plate-shaped body. Thus, in this example, the palm support face 2, the finger support face 3 and the forearm support face 9 are designed as a substantially plate-shaped body. The substantially plate-shaped body can have a thickness of, for instance, 1-10 mm, preferably 2-7 mm, more preferably 3-5 mm. The substantially plate-shaped body can be manufactured from metal and/or plastic.

In the example of FIG. 7a, the forearm support face 9 is of lowered design in the width direction thereof near the middle thereof. This provides the advantage that the aid abuts comfortably against the forearm, and can be firmly connected with the forearm.

In the example of FIG. 7a, the aid 1 is provided with a recess 10 for leaving the thumb substantially clear. Thus, the thumb remains movable if the aid, possibly together with plaster, has been applied to the hand and/or forearm. In this way, the user can continue to use the thumb for performing tasks, such as grasping things, and the thumb is not unnecessarily immobilized, which could adversely affect a healing process.

FIG. 7b shows an alternative advanced embodiment of the aid 1 according to the invention. In this embodiment, the palm support face 2 is arranged for supporting the inner side of the palm under substantially exclusively the fourth and fifth metacarpals associated with the ring finger and the little finger, respectively, and the finger support face 3 is arranged for supporting substantially exclusively the little finger and ring finger at least to a point near the proximal interphalangeal joints, in this example to a point beyond the proximal interphalangeal joint. In this example, the aid 1 is designed in one piece, i.e. of monolithic design. In this example, the aid 1 is designed as a substantially plate-shaped body.

In the example of FIG. 7b, the aid 1 is furthermore provided with an upstanding edge 11 which extends upwards on a side edge of the finger support face 3 and the palm support face 2. The upstanding edge extends such that, in use, it abuts against a side of the palm and the little finger. This provides the advantage that the little finger and the associated metacarpal is efficiently immobilized. It is also possible that the upstanding edge additionally extends along the forearm.

In the aid according to the present invention, the angle between the palm support face and the horizontal (the base of the aid) is suitably between 30° and 90°, preferably between 40° and 85°, in particular between 40° and 80°.

The invention is not limited in any way to the examples described above. For instance, it is possible for the aid to be manufactured by injection molding, thermoforming, deep-drawing and/or deep-pressing.

It is also possible that the aid is manufactured in a plurality of discrete sizes, for instance large (L), medium (M) and small (S), which sizes have been chosen such as to allow a large number of users to make use of one of the aids. Possibly, the different sizes are indicated by a color coding, for instance by the color of the aid.

Furthermore, it is possible that a side of the aid remote from the hand is made of slightly rough design, so that the plaster, if it is applied on the side of the aid remote from the hand, can properly adhere to the aid.

All these variants are understood to fall within the framework of the invention.

1. An aid for relative positioning of a palm of a hand, and associated fingers and forearm, which aid comprises a palm support face for laying against it an inner side of the palm and a finger support face for laying against it the fingers at least from a point near the metacarpophalangeal joints to a point near the proximal interphalangeal joints.

2. An aid according to claim 1, wherein a transition of the palm support face and the finger support face includes a first knuckle support surface and a second knuckle support surface situated remote from the first knuckle support surface, wherein the first knuckle support surface of the transition is situated higher than the second surface.

3. An aid according to claim 2, wherein between the first knuckle support surface and the second knuckle support surface of the transition a third knuckle support surface is provided, which is situated higher than the first knuckle support surface.

4. An aid according to claim 1, wherein the palm. support face is arranged for supporting substantially the whole palm, and the finger support face is arranged for supporting the four fingers at least to a point near the proximal interphalangeal joints.

5. An aid according to claim 1, wherein the palm support face is arranged for supporting the inner side of the palm under substantially exclusively the fourth and fifth metacarpals, and the finger support face is arranged for supporting substantially exclusively the little finger and ring finger at least to a point near the proximal interphalangeal joints.

6. An aid according to claim 1, wherein the aid is arranged for leaving the thumb clear.

7. An aid according to claim 3, wherein the third knuckle support surface is positioned such that, in use, it is placed under the metacarpophalangeal joint of the middle finger.

8. An aid according to claim 2, wherein the first knuckle support surface of the transition is positioned such that, in use, it is placed under the metacarpophalangeal joint of the little finger.

9. An aid according to claim 1, wherein the palm support face and the finger support face include a mutual angle of 75-110°, preferably 85-95°.

10. An aid according to claim 1, wherein the palm support face is made of convex design over at least a part of the surface thereof

11. An aid according to claim 1, wherein the finger support face is made of convex design over at least a part of the surface thereof

12. An aid according to claim 1, wherein the aid, remote from the transition of the palm support face and the finger support face, on the side of the palm support face, possesses a forearm support face for positioning the forearm against it.

13. An aid according to claim 12, wherein the palm support face and the forearm support face include a mutual angle of 65-100°, preferably 65-85°.

14. An aid according to claim 12, wherein the forearm support face is of lowered design in the width direction near the middle thereof.

15. An aid according to claim 1, wherein the aid is designed in one piece.

16. An aid according to claim 1, wherein the palm support face, the finger support face and possibly the forearm support face are designed as a substantially plate-shaped body.

17. An aid according to claim 16, wherein the aid is designed as a substantially plate-shaped body.

18. An aid according to claim 16, wherein the substantially plate-shaped body is of a thickness of 1-10 mm, preferably 2-7 mm, more preferably 3-5 mm.

19. An aid according to claim 16, wherein the substantially plate-shaped body is manufactured from metal and/or plastic.

20. An aid according to claim 1, wherein the aid is manufactured through injection molding, thermoforming, deep-drawing, and/or deep-pressing.

21. An aid according to claim 1, wherein the aid is designed as a disposable article.

22. An aid according to claim 1, wherein the aid is arranged to remain connected with the hand during a healing process.

23. An assembly of an aid according to claim 1 and a plaster sleeve, wherein, in use, the plaster sleeve at least partly encloses the palm, fingers and possibly the forearm, and wherein the plaster sleeve is placed at least partly between the aid and the palm, fingers and forearm.

24. An assembly of an aid according to claim 1 and a plaster sleeve, wherein, in use, the plaster sleeve at least partly encloses the palm, fingers and possibly the forearm, and wherein the aid is placed at least partly between the plaster sleeve and the palm, fingers and forearm.

25. An assembly according to claim 23, wherein the plaster sleeve comprises a natural plaster or a synthetic plaster.

26. A method for manufacturing an aid according to claim 16, comprising providing a substantially planar plate-shaped body and then deforming the substantially plate-shaped body.

27. A method for applying plaster to a hand, comprising:
applying a plaster sleeve, at least partly, around the hand,
laying an aid according to claim 1 against the hand, and
allowing the plaster sleeve to set while the plaster sleeve lies against the aid.

28. A method according to claim 27, wherein during setting the aid lies against an outer side of the plaster sleeve remote from the hand.

29. A method according to claim 27, wherein during setting the aid is placed between the hand and the plaster sleeve.

30. A method according to claim 29, wherein the plaster sleeve is provided, at least partly, around the aid and the hand, such that the aid, by means of the plaster sleeve, is permanently connected with the hand.

31. A method according to claim 29, further comprising placing a cushion, such as cotton wadding between the aid and the hand.

32. An aid for relative positioning of a palm of a hand, fingers, and forearm, which are provided with a non-set plaster, which aid comprises a first support face for laying against it the inner side of the palm provided with plaster, and a second support face for laying against it fingers provided with plaster at least beyond the knuckles of the hand, wherein the first support face and the second support face include a mutual angle of 75-110°.

33. An aid according to claim 32, wherein the first support face is of convex design over at least a part of the surface thereof.

34. An aid according to claim 32, wherein the transition of the first support face and the second support face possesses a first surface and a second surface situated remote from the first surface, wherein the first surface of the transition is situated higher than the second surface.

35. An aid according to claim 34, wherein between the first surface and the second surface of the transition a third surface is provided, which is situated higher than the first surface.

36. An aid according to claim 32, wherein the first support face and the second support face include a mutual angle of 85-95°.

37. An aid according to claim 32, wherein the aid, remote from the transition of the first support face and the second support face, on the side of the first support face, possesses a third support face for positioning the forearm, the first support face and the third support face including a mutual angle of 65-100°.

38. An aid according to claim 37, wherein the first support face and the third support face include a mutual angle of 75-95°.

39. An aid according to claim 37, wherein the third support face is of lowered design in the longitudinal direction thereof near the middle thereof
